(19) United States
(12) Patent Application Publication
(10) Pub. No.: US 2014/0348869 A1
(43) Pub. Date: Nov. 27, 2014

(54) MODIFIED MENINGOCOCCAL FHBP POLYPEPTIDES

(71) Applicant: Novartis AG, Basel (CH)

(72) Inventors: Lucia BANCI, Sesto Fiorentino (IT); Francesca CANTINI, Sesto Fiorentino (IT); Sara DRAGONETTI, Sesto Fiorentino (IT); Maria Antonietta GENTILE, Siena (IT); Daniele VEGGI, Siena (IT); Maria SCARSELLI, Siena (IT); Mariagrazia PIZZA, Siena (IT)

(21) Appl. No.: 14/335,788

(22) Filed: Jul. 18, 2014

Related U.S. Application Data
(63) Continuation of application No. 13/504,476, filed on Oct. 10, 2012.
(60) Provisional application No. 61/279,977, filed on Oct. 27, 2009.

(51) Int. Cl. C07K 14/22; A61K 39/09; A61K 39/095

(52) U.S. Cl. CPC C07K 14/22; A61K 39/095; A61K 39/092
USPC 424/190.1; 530/359; 536/23.7; 435/320.1; 435/252.3

(57) ABSTRACT

The factor H binding activity of meningococcal fHBP can be uncoupled from its bactericidal sensitivity. NMR studies have identified various amino acid residues involved in the fHBP/fH interaction and one or more of these residues is modified in a fHBP to reduce or eliminate its ability to bind to fH.

### CROSS REFERENCE TO RELATED APPLICATIONS

This application is a Continuation of U.S. patent application Ser. No. 13/504,476, claiming an international filing date of Oct. 27, 2010; which is a U.S. National Phase International Patent Application No. PCT/IB2010/054865, filed Oct. 27, 2010; which claims the benefit of U.S. Provisional Patent Application No. 61/279,977, filed Oct. 27, 2009; all of which are hereby incorporated by reference in the present disclosure in their entirety.

### TECHNICAL FIELD

This invention is in the field of immunisation and, in particular, immunisation against diseases caused by pathogenic bacteria in the genus Neisseria, such as N. meningitidis (meningococcus).

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 223002124701SeqList.txt, date recorded: Jun. 27, 2014, size: 95 KB).

### BACKGROUND ART

Neisseria meningitidis is a Gram-negative encapsulated bacterium which colonises the upper respiratory tract of approximately 10% of human population. Although polysaccharide and conjugate vaccines are available against serogroups A, C, W135 and Y, this approach cannot be applied to serogroup B because the capsular polysaccharide is a polymer of polysialic acid, which is a self antigen in humans. To develop a vaccine against serogroup B, surface-exposed proteins contained in outer membrane vesicles (OMVs) have been used. These vaccines elicit serum bactericidal antibody responses and protect against disease, but they fail to induce cross-strain protection [1]. Some workers are therefore focusing on specific meningococcal antigens for use in vaccines [2]. One such antigen is the meningococcal factor H binding protein (fHBP), also known as protein ‘741’ [SEQ IDs 2535 & 2536 in ref. 3; SEQ ID 1 herein], ‘NMB1870’, ‘GNA1870’ [refs. 4-6, following ref 2], ‘P2086’, ‘LP2086’ or ‘ORF2086’ [7-9]. This lipoprotein is expressed across all meningococcal serogroups and has been found in multiple meningococcal strains. fHBP sequences have been grouped into three families [4] (referred to herein as families I, II & III), and it has been found that serum raised against a given family is bactericidal within the same family, but is not active against strains which express one of the other two families i.e. there is intra-family cross-protection, but not inter-family cross-protection.

### DISCLOSURE OF THE INVENTION

Uncoupling fHBP's ability to bind to fH from its immunogenicity could given an improved antigen. For example, important epitopes on fHBP's surface could be hidden from the immune system in vivo following fH binding. Conversely, high affinity binding of a host protein to a vaccine component could lead to unintended post-vaccination consequences in some subjects. Thus it is an object of the invention to provide modified fHBPs which, compared to wild-type fHBPs, show reduced binding to fH while maintaining the ability to elicit bactericidal anti-fHBP antibodies. Reference 10 already identified various residues important in the fHBP/fH interaction. For example, mutation of two wild-type glutamate residues reduced the protein's affinity for fH by two orders of magnitude. Reference 10 did not disclose, however, the impact of these changes on the fHBP's immunogenic activity. As shown herein, though, bacteria expressing the double-Glu mutant are sensitive to bactericidal antibodies elicited by wild-type fHBP. Thus the fH-binding activity of fHBP can be uncoupled from its bactericidal sensitivity.

Full-length fHBP has the following amino acid sequence (SEQ ID NO: 1) in strain MC58:

MNRTAFCCLSLTTALILTACSSGGGGVAADIGAGLADALTAPLDHKDKGL
QSLTLDQSVRKNEKLKLAAQGAEKTYGNGDSLNTGKLKNDKVSRFDFIRQ
IEVDGQLITLESGEFQVYKQSHSALTAFQTEQIQDSEHSGKMVAKRQFRI
GDIAGEHTSFDKLPEGGRATYRGTAFGSDDAGGKLTYTIDFAAKQGNGKI
EHLKSPELNVDLAAADIKPDGKRHAVISGSVLYNQAEKGSYSLGIFGGKA
QEVAGSAEVKTVNGIRHIGLAAKQ

This sequence is in fHBP family 1. The mature lipoprotein lacks the first 19 amino acids of SEQ ID NO: 1 (SEQ ID NO: 4), and the AG form of fHBP lacks the first 26 amino acids (SEQ ID NO: 7).

Full-length MHBP has the following amino acid sequence (SEQ ID NO: 2) in strain 2996:

MNRTAFCCLSLTAALILTACSSGGGGVAADIGAGLADALTAPLDHKDKSL
QSLTLDQSVRKNEKLKLAAQGAEKTYGNGDSLNTGKLKNDKVSRFDFIRQ
IEVDGQLITLESGEFQIYKQDHSAVVALQIEKINNPDKIDSLINQRSFLV
SGLGGEHTAFNQLPDGKAEYHGKAFSSDDAGGKLTYTIDFAAKQGHGKIE
HLKTPEQNVELAAAELKADEKSHAVILGDTRYGSEEKGTYHLALFGDRAQ
EIAGSATVKIGEKVHEIGIAGKQ

This sequence is in fHBP family II. The mature lipoprotein lacks the first 19 amino acids of SEQ ID NO: 1 (SEQ ID NO: 5), and the ΔG form of fHBP lacks the first 26 amino acids (SEQ ID NO: 8).

Full-length fH-BP has the following amino acid sequence (SEQ ID NO: 3) in strain M1239:

MNRTAFCCLSLTTALILTACSSGGGGSGGGGVAADIGTGLADALTAPLDH
KDKGLKSLTLEDSIPQNGTLTLSAQGAEKTFKAGDKDNSLNTGKLKNDKI
SRFDFVQKIEVDGQTITLASGEFQIYKQNHSAVVALQIEKINNPDKTDSL
INQRSFLVSGLGGEHTAFNQLPGGKAEYHGKAFSSDDPNGRLHYSIDFTK
KQGYGRIEHLKTLEQNVELAAAELKADEKSHAVILGDTRYGSEEKGTYHL
ALFGDRAQEIAGSATVKIGEKVHEIGIAGKQ

This sequence is in fHBP family III. The mature lipoprotein lacks the first 19 amino acids of SEQ ID NO: 1 (SEQ ID NO: 6), and the AG form of fHBP lacks the first 31 amino acids (SEQ ID NO: 9).

NMR studies have identified various amino acid residues involved in the fHBP/fH interaction. Thus one or more of the following residues, numbered according to each of SEQ ID NO.: 4, 5 and 6, may be modified in order to inhibit the fH/fHBP interaction:

| SEQ ID NO: 4 | SEQ ID NO: 5 | SEQ NO: 6 | |
|---|---|---|---|
| Asp-37 | Asp-37 | Glu-42 | * |
| Asn-43 | Asn-43 | Asn-48 | |
| Lys-45 | Lys-45 | Thr-50 | * |
| Thr-56 | Thr-56 | Thr-61 | |
| Glu-83 | Glu-83 | Glu-91 | * |
| Glu-95 | Glu-95 | Glu-103 | * |
| Glu-112 | Glu-112 | Glu-120 | * |
| Asp-116 | Asn-116 | Asn-124 | |
| His-119 | Lys-119 | Lys-127 | |
| Lys-122 | Ser-122 | Ser-130 | |
| Val-124 | Ile-124 | Ile-132 | * |
| Arg-127 | Arg-127 | Arg-135 | * |
| Thr-139 | Thr-139 | Thr-147 | * |
| Phe-141 | Phe-141 | Phe-149 | * |
| Asp-142 | Asn-142 | Asn-150 | |
| Lys-143 | Gln-143 | Gln-151 | * |
| Ile-198 | Leu-197 | Leu-205 | |
| Ser-211 | Asp-210 | Asp-218 | |
| Leu-213 | Arg-212 | Arg-220 | * |
| Lys-219 | Lys-218 | Lys-226 | |
| Ser-221 | Thr-220 | Thr-228 | |
| Lys-241 | Lys-240 | Lys-248 | |

The rows marked with a * are preferred residues because they were not present in the fH binding site defined by the X-ray study in reference 10. Without wishing to be bound by theory, these extra residues could have been identified due to (i) the more natural conditions which exist during NMR experiments compared to X-ray crystals and/or (ii) the inclusion of fH domain 5 in the NMR study.

Reference 11 discloses fHBP proteins which are modified at residues which interact with fH. Specific amino acid residues which are suggested for modification include 38, 41, 42, 43, 44, 80, 82, 84, 85, 89, 91, 92, 115, 116, 117, 118, 119, 120, 126, 128, 129, 130, 131, 134, 197, 199, 201, 202, 203, 207, 209, 218, 220, 221, 223, 224, 237, 239, 241, 246, and 248 (numbered according to SEQ ID NO: 4, which is 65 less than reference 11's own numbering). The two preferred residues in reference 11 are Glu-218 and Glu-239 as mutation of these residues to alanine gave a protein with “an almost complete ablation of factor H binding”. The residues listed in reference 11 overlap with the residues given herein (referring only to SEQ ID NO: 4) as follows: 43, 116, 119, 221 and 241. In some embodiments of the present invention, the polypeptide does not include SEQ ID NO: 35.

The invention therefore provides a polypeptide comprising an amino acid sequence: (a) which has at least k % identity to any one of SEQ ID NOs: 4, 5 or 6, and/or comprises a fragment of SEQ ID NO: 4, 5 or 6; but (b) wherein one or more of the amino acid residues listed in the above table has been either deleted or substituted by a different amino acid. A fragment of (a) will include the relevant table residue of (b). The polypeptide can, after administration to a host animal, elicit antibodies which can recognise a wild-type meningococcal polypeptide consisting of SEQ ID NO: 4, 5 or 6. The polypeptide has, under the same experimental conditions, a lower affinity for human factor H than the same polypeptide but without the modification(s) of (b).

Thus the invention also provides a polypeptide comprising an amino acid sequence: (a) which has at least A % identity to SEQ ID NO: 4 and/or comprises a fragment of SEQ ID NO: 4; but (b) wherein one or more of the amino acid residues listed in the above table has been either deleted or substituted by a different amino acid. The polypeptide can, after administration to a host animal, elicit antibodies which can recognise a wild-type meningococcal polypeptide consisting of SEQ ID NO: 4. The polypeptide has, under the same experimental conditions, a lower affinity for human fH than the same polypeptide but without the modification(s) of (b). The polypeptide has, under the same experimental conditions, a lower affinity for human fH than a wild-type meningococcal polypeptide consisting of SEQ ID NO: 4.

Similarly, the invention provides a polypeptide comprising an amino acid sequence: (a) which has at least k % identity to SEQ ID NO: 5 and/or comprises a fragment of SEQ ID NO: 5; but (b) wherein one or more of the amino acid residues listed in the above table has been either deleted or substituted by a different amino acid. The polypeptide can, after administration to a host animal, elicit antibodies which can recognise a wild-type meningococcal polypeptide consisting of SEQ ID NO: 5. The polypeptide has, under the same experimental conditions, a lower affinity for human fH than the same polypeptide but without the modification(s) of (b). The polypeptide has, under the same experimental conditions, a lower affinity for human fH than a wild-type meningococcal polypeptide consisting of SEQ ID NO: 5.

Similarly, the invention provides a polypeptide comprising an amino acid sequence: (a) which has at least k % identity to SEQ ID NO: 6 and/or comprises a fragment of SEQ ID NO: 6; but (b) wherein one or more of the amino acid residues listed in the above table has been either deleted or substituted by a different amino acid. The polypeptide can, after administration to a host animal, elicit antibodies which can recognise a wild-type meningococcal polypeptide consisting of SEQ ID NO: 6. The polypeptide has, under the same experimental conditions, a lower affinity for human fH than the same polypeptide but without the modification(s) of (b). The polypeptide has, under the same experimental conditions, a lower affinity for human fH than a wild-type meningococcal polypeptide consisting of SEQ ID NO: 6.

The value of k may be selected from 85, 86, 87, 88, 89, 90, 91, 92, 93, 94, 95, 96, 97, 98, 99 or more. It is preferably 90 or more.

A fragment of (a) will include the relevant table residue of (b), but that residue will be deleted or substituted when compared to the relevant SEQ ID residue. A fragment will generally be at least 7 amino acids long e.g. 8, 10, 12, 14, 16, 18, 20, 22, 24, 26, 28, 30, 32, 24, 26, 28, 40, 45, 50, 55, 60 contiguous amino acids or more. The fragment will typically include an epitope from the SEQ ID.

In some preferred embodiments, the polypeptide of the invention is truncated relative to SEQ ID NO: 4, 5 or 6 e.g. truncated at the N-terminus up to and including the poly-glycine sequence (as in SEQ ID NOs: 7, 8 and 9). Thus the polypeptide may comprise an amino sequence with at least k % identity to any one of SEQ ID NOs: 7, 8 or 9 with modification of one or more of the amino acid residues listed in the above table.

The reduction in fH affinity is ideally at least 2-fold lower e.g. ≧5-fold, ≧10-fold, ≧50-fold, ≧100-fold, etc., and fH binding may be totally eliminated. The affinity of a fH/fHBP interaction can suitably be assessed using the methods and reagents disclosed in reference 10 e.g. by surface plasmon resonance using immobilised fH and 50 nM of soluble fHBP (or vice versa).

The invention also provides a method for designing a modified fHBP amino acid sequence comprising steps of: (i) providing a starting amino acid sequence, wherein a protein consisting of or comprising the starting amino acid sequence can bind to human factor H; (ii) identifying within the starting amino acid sequence an amino acid residue which, using a pairwise alignment algorithm, aligns with a residue in SEQ ID NO: 4, 5 or 6 shown in the above table; (iii) either deleting the amino acid identified in step (ii), or replacing it with a different amino acid, thereby providing the modified fHBP amino acid sequence. Steps (ii) and (iii) can be repeated one or more times. A protein consisting of or comprising the starting amino acid sequence can bind to human factor H with a higher affinity than the same protein after performing the method. The starting amino acid sequence can be a wild-type of sequence e.g. it can be any of the wild-type or modified or artificial fHBP amino acid sequences disclosed in references 4, 5, 7, 8, 9, 195, 196, 197, 198, 199, 200 & 201. For example, the starting amino acid sequence can be any of SEQ ID NOs: 1 to 9 or 20 to 22 herein.

The invention also provides a polypeptide comprising a modified fHBP amino acid sequence designed by this method. The polypeptide is immunogenic and can bind to human factor H.

### Modifications

Polypeptides of the invention include a modification at one or more of the amino acid residues listed in the table e.g. at 2, 3, 4, 5 or more of the residues.

A residue indicated in the table is either deleted or is substituted by a different amino acid. For example, Asp-37 can be substituted by any of the other 19 naturally-occurring amino acids. When a substitution is made, the replacement amino acid in some embodiments may be a simple amino acid such as glycine or alanine. In other embodiments, the replacement amino acid is non-conservative. Conservative substitutions may be made within the following four groups: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Substitution by alanine is preferred in some embodiments.

Where more than one modification is made, the modifications may be selected from the following groups A to D:
A: residues 112, 116, 119, 122, and/or 127.
B: residues 43, 45, 56, and/or 83.
C: residues 211, 219, 221, and/or 241.
D: residues 139, 141, 142, 143, and/or 198.
   Thus, for example, if residue 112 is to be modified then a preferred second residue for modification would be 116, 119, 122 or 127, and if residue 43 is to be modified then a preferred second residue for modification would be 45, 56, or 83, etc.

### Siderophore Binding

The fHBP shows structural homology with siderocalin. Siderocalin can bind to enterobactin, a bacterial siderophore. As shown herein, fHBP can also bind to enterobactin. Thus the invention provides a complex of a Neisserial (e.g. meningococcal) fHBP and a siderophore.

Siderophores are usually classified by the ligands therein which are able to chelate iron. They may be catecholates, hydroxamates or carboxylates. In some embodiments the siderophore is not citric acid. The siderophore may be selected from ferrichrome, desferrioxamine B, desferrioxamine E, fusarinine C, ornibactin, enterobactin, bacillibactin, vibriobactin, azotobactin, pyoverdine, aerobactin, salmochelin or yersiniabactin. It is preferably salmochelin or, more preferably, enterobactin.

The siderophore will usually include a chelated iron (Fe³⁺) ion, such as a hexadentate octahedral complex of Fe³⁺. Rather than iron, however, in some embodiments the siderophore may include a chelated ion of aluminium, gallium, chromium, copper, zinc, lead, manganese, cadmium, vanadium, indium, plutonium, or uranium.

The invention also provides a polypeptide comprising an amino acid sequence: (a) which has at least k % identity to any one of SEQ ID NOs: 4, 5 or 6, and/or comprises a fragment of SEQ ID NO: 4, 5 or 6; (b) can, after administration to a host animal, elicit antibodies which can recognise a wild-type meningococcal polypeptide consisting of SEQ ID NO: 4, 5 or 6; but (c) does not bind to enterobactin. The value of k and the length of a fragment are as defined above.

This polypeptide can, compared to SEQ ID NO: 4, have a mutation at one or more of amino acids 102, 136-138, 148-154, 166, 205, 230 and 254. Thus the amino acid in the polypeptide which aligns with one or more of these residues in SEQ ID NO: 4 using a pairwise alignment algorithm is different from the amino acid residue in SEQ ID NO: 4. For instance, Lys-254 can be replaced by an non-Lys residue (e.g. by alanine). Thus the invention provides, for example, a polypeptide comprising any of SEQ ID NOs: 29, 30, 31 and 32.

The invention also provides a method for designing a modified fHBP amino acid sequence comprising steps of: (i) providing a starting amino acid sequence, wherein a protein consisting of or comprising the starting amino acid sequence can bind to human factor H and to a siderophore; (ii) identifying within the starting amino acid sequence an amino acid residue which interacts with a siderophore; (iii) either deleting the amino acid identified in step (ii), or replacing it with a different amino acid, thereby providing the modified fHBP amino acid sequence. The starting amino acid sequence can have at least k % identity to any one of SEQ ID NOs: 4, 5 or 6.

### Polypeptides

Polypeptides of the invention can be prepared by various means e.g. by chemical synthesis (at least in part), by digesting longer polypeptides using proteases, by translation from RNA, by purification from cell culture (e.g. from recombinant expression or from N. meningitidis culture), etc. Heterologous expression in an E. coli host is a preferred expression route.

fHBP is naturally a lipoprotein in N. meningitidis. It has also been found to be lipidated when expressed in E. coli with its native leader sequence. Polypeptides of the invention may have a N-terminus cysteine residue, which may be lipidated e.g. comprising a palmitoyl group, usually forming tripalmitoyl-S-glyceryl-cysteine. In other embodiments the polypeptides are not lipidated.

Polypeptides are preferably prepared in substantially pure or substantially isolated form (i.e. substantially free from other Neisserial or host cell polypeptides) or substantially isolated form. In general, the polypeptides are provided in a non-naturally occurring environment e.g. they are separated from their naturally-occurring environment. In certain embodiments, the subject polypeptide is present in a composition that is enriched for the polypeptide as compared to a control. As such, purified polypeptide is provided, whereby purified is meant that the polypeptide is present in a composition that is substantially free of other expressed polypeptides, where by substantially free is meant that less than 90%, usually less than 60% and more usually less than 50% of the composition is made up of other expressed polypeptides.

Polypeptides can take various forms (e.g. native, fusions, glycosylated, non-glycosylated, lipidated, disulfide bridges, etc.).

SEQ ID NOs 4 to 9 do not include a N-terminus methionine. If a polypeptide of the invention is produced by translation in a biological host then a start codon is required, which will provide a N-terminus methionine in most hosts. Thus a polypeptide of the invention will, at least at a nascent stage, include a methionine residue upstream of said SEQ ID NO sequence.

In some embodiments the polypeptide has a single methionine at the N-terminus immediately followed by the SEQ ID NO sequence; in other embodiments a longer upstream sequence may be used. Such an upstream sequence may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art e.g. the native upstream sequences present in SEQ ID NOs: 1, 2 and 3.

A polypeptide of the invention may also include amino acids downstream of the final amino acid of the SEQ ID NO sequences. Such C-terminal extensions may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. His, where n=3, 4, 5, 6, 7, 8, 9, 10 or more), or sequences which enhance polypeptide stability. Other suitable C-terminal amino acid sequences will be apparent to those skilled in the art.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

Polypeptides of the invention may be attached or immobilised to a solid support.

Polypeptides of the invention may comprise a detectable label e.g. a radioactive label, a fluorescent label, or a biotin label. This is particularly useful in immunoassay techniques.

As disclosed in reference 199, fHBP can be split into three domains, referred to as A, B and C. Taking SEQ ID NO: 1, the three domains are (A) 1-119, (B) 120-183 and (C) 184-274:

MNRTAFCCLSLTTALILTACSSGGGGVAADIGAGLADALTAPLDHKDKGL
QSLTLDQSVRKNEKLKLAAQGAEKTYGNGDSLNTGKLKNDKVSRFDFIRQ
IEVDGQLITLESGEFQVYKQSHSALTAFQTEQIQDSEHSGKMVAKRQFRI
GDIAGEHTSFDKLPEGGRATYRGTAFGSDDAGGKLTYTIDFAAKQGNGKI
EHLKSPELNVDLAAADIKPDGKRHAVISGSVLYNQAEKGSYSLGIFGGKA
QEVAGSAEVKTVNGIRHIGLAAKQ

The mature form of domain ‘A’, from Cys-20 at its N-terminus to Lys-119, is called ‘Aₘₐₜᵤᵣₑ’.

Multiple fHBP sequences are known and these can readily be aligned using standard methods. By such alignments the skilled person can identify (a) domains ‘A’ (and ‘Aₘₐₜᵤᵣₑ’). ‘B’ and ‘C’ in any given fHBP sequence by comparison to the coordinates in the MC58 sequence, and (b) single residues in multiple fHBP sequences e.g. for identifying substitutions. For case of reference, however, the domains are defined below:
Domain ‘A’ in a given fHBP sequence is the fragment of that sequence which, when aligned to SEQ ID NO: 1 using a pairwise alignment algorithm, starts with the amino acid aligned to Met-1 of SEQ ID NO: 1 and ends with the amino acid aligned to Lys-119 of SEQ ID NO: 1.
Domain ‘Aₘₐₜᵤᵣₑ’ in a given fHBP sequence is the fragment of that sequence which, when aligned to SEQ ID NO: 1 using a pairwise alignment algorithm, starts with the amino acid aligned to Cys-20 of SEQ ID NO: 1 and ends with the amino acid aligned to Lys-119 of SEQ ID NO: 1.
Domain ‘B’ in a given fHBP sequence is the fragment of that sequence which, when aligned to SEQ ID NO: 1 using a pairwise alignment algorithm, starts with the amino acid aligned to Gln-120 of SEQ ID NO: 1 and ends with the amino acid aligned to Gly-183 of SEQ ID NO: 1.
Domain ‘C’ in a given fHBP sequence is the fragment of that sequence which, when aligned to SEQ ID NO: 1 using a pairwise alignment algorithm, starts with the amino acid aligned to Lys-184 of SEQ ID NO: 1 and ends with the amino acid aligned to Gln-274 of SEQ ID NO: 1.

The preferred pairwise alignment algorithm for defining the domains is the Needleman-Wunsch global alignment algorithm [12], using default parameters (e.g. with Gap opening penalty=10.0, and with Gap extension penalty=0.5, using the EBLOSUM62 scoring matrix). This algorithm is conveniently implemented in the needle tool in the EMBOSS package [13].

In some embodiments, a polypeptide of the invention is truncated to remove its domain A i.e. domain A is omitted from a SEQ ID.

In some embodiments, a polypeptide comprises an amino acid sequence as described above, except that up to 10 amino acids (i.e. 1, 2, 3, 4, 5, 6, 7, 8, 9 or 10) at the N-terminus and/or up to 10 amino acids (i.e. 1, 2, 3, 4, 5, 6, 7, 8, 9 or 10) at the C-terminus are deleted.

### Nucleic Acids

The invention provides nucleic acid encoding a polypeptide of the invention as defined above.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acids of the invention can take various forms e.g. single-stranded, double-stranded, vectors, primers, probes, labelled, unlabelled, etc.

Nucleic acids of the invention are preferably in isolated or substantially isolated form.

The term “nucleic acid” includes DNA and RNA, and also their analogues, such as those containing modified backbones, and also peptide nucleic acids (PNA), etc.

Nucleic acid according to the invention may be labelled e.g. with a radioactive or fluorescent label.

The invention also provides vectors (such as plasmids) comprising nucleotide sequences of the invention (e.g. cloning or expression vectors, such as those suitable for nucleic acid immunisation) and host cells transformed with such vectors.

### Bactericidal Responses

Preferred polypeptides of the invention can elicit antibody responses that are bactericidal against meningococci. Bactericidal antibody responses are conveniently measured in mice and are a standard indicator of vaccine efficacy (e.g. see end-note 14 of reference 2). Polypeptides of the invention can preferably elicit an antibody response which is bactericidal against at least one N. meningitidis strain in at least one of the following three groups of strains:
(I) MC58, gb185 (=M01-240185), m4030, m2197, m2937, iss1001, NZ394/98, 67/00, 93/114, bz198, m1390, nge28, lnp17592, 00-241341, f6124, 205900, m198/172, bz133, gb149 (=M01-240149), nm008, nm092, 30/00, 39/99, 72/00, 95330, bz169, bz83, cu385, h44/76, m1590, m2934, m2969, m3370, m4215, m4318, n44/89, 14847.
(II) 961-5945, 2996, 96217, 312294, 11327, a22, gb013 (=M01-240013), e32, m1090, m4287, 860800, 599, 95N477, 90-18311, c11, m986, m2671, 1000, m1096, m3279, bz232, dk353, m3697, ngh38, L93/4286.
(III) M1239, 16889, gb355 (=M01-240355), m3369, m3813, ngp165.

For example, a polypeptide may elicit a bactericidal response effective against serogroup B N. meningitidis strains MC58, gb185 and NZ394/98.

### Immunisation

Polypeptides of the invention may be used as the active ingredient of immunogenic compositions, and so the invention provides an immunogenic composition comprising a polypeptide of the invention.

The invention also provides a method for raising an antibody response in a mammal, comprising administering an immunogenic composition of the invention to the mammal. The antibody response is preferably a protective and/or bactericidal antibody response. The invention also provides polypeptides of the invention for use in such methods.

The invention also provides a method for protecting a mammal against a Neisserial (e.g. meningococcal) infection, comprising administering to the mammal an immunogenic composition of the invention.

The invention provides polypeptides of the invention for use as medicaments (e.g. as immunogenic compositions or as vaccines) or as diagnostic reagents. It also provides the use of nucleic acid, polypeptide, or antibody of the invention in the manufacture of a medicament for preventing Neisserial (e.g. meningococcal) infection in a mammal.

The mammal is preferably a human. The human may be an adult or, preferably, a child. Where the vaccine is for prophylactic use, the human is preferably a child (e.g. a toddler or infant); where the vaccine is for therapeutic use, the human is preferably an adult. A vaccine intended for children may also be administered to adults e.g. to assess safety, dosage, immunogenicity, etc.

The uses and methods are particularly useful for preventing/treating diseases including, but not limited to, meningitis (particularly bacterial, such as meningococcal, meningitis) and bacteremia.

Efficacy of therapeutic treatment can be tested by monitoring Neisserial infection after administration of the composition of the invention. Efficacy of prophylactic treatment can be tested by monitoring immune responses against fHBP after administration of the composition.

Immunogenicity of compositions of the invention can be determined by administering them to test subjects (e.g. children 12-16 months age, or animal models [14]) and then determining standard parameters including serum bactericidal antibodies (SBA) and ELISA titres (GMT). These immune responses will generally be determined around 4 weeks after administration of the composition, and compared to values determined before administration of the composition. A SBA increase of at least 4-fold or 8-fold is preferred. Where more than one dose of the composition is administered, more than one post-administration determination may be made.

Preferred compositions of the invention can confer an antibody titre in a patient that is superior to the criterion for seroprotection for each antigenic component for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is about 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

The immunogenic composition of the invention will generally include a pharmaceutically acceptable carrier, which can be any substance that does not itself induce the production of antibodies harmful to the patient receiving the composition, and which can be administered without undue toxicity. Pharmaceutically acceptable carriers can include liquids such as water, saline, glycerol and ethanol. Auxiliary substances, such as wetting or emulsifying agents, pH buffering substances, and the like, can also be present in such vehicles. A thorough discussion of suitable carriers is available in ref. 15.

Neisserial infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be prepared for topical administration e.g. as an ointment, cream or powder. The composition be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops.

The composition is preferably sterile. It is preferably pyrogen-free. It is preferably buffered e.g. at between pH 6 and pH 8, generally around pH 7. Where a composition comprises an aluminium hydroxide salt, it is preferred to use a histidine buffer [16]. Compositions of the invention may be isotonic with respect to humans.

Immunogenic compositions comprise an immunologically effective amount of immunogen, as well as any other of other specified components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Dosage treatment may be a single dose schedule or a multiple dose schedule (e.g. including booster doses). The composition may be administered in conjunction with other immunoregulatory agents.

Adjuvants which may be used in compositions of the invention include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 17, or mixtures of different mineral compounds, with the compounds taking any suitable form (e.g. gel, crystalline, amorphous, etc.), and with adsorption being preferred. The mineral containing compositions may also be formulated as a particle of metal salt [18].

A useful aluminium phosphate adjuvant is amorphous aluminium hydroxyphosphate with PO₄/Al molar ratio between 0.84 and 0.92, included at 0.6 mg Al³⁺/ml.

### B. Oil Emulsions

Oil emulsion compositions suitable for use as adjuvants in the invention include squalene-in-water emulsions, such as MF59 [Chapter 10 of ref. 17; see also ref. 19](5% Squalene, 0.5% Tween 80, and 0.5% Span 85, formulated into submicron particles using a microfluidizer). Complete Freund's adjuvant (CFA) and incomplete Freund's adjuvant (IFA) may also be used.

Useful oil-in-water emulsions typically include at least one oil and at least one surfactant, with the oil(s) and surfactant(s) being biodegradable (metabolisable) and biocompatible. The oil droplets in the emulsion are generally less than 1 μm in diameter, with these small sizes being achieved with a microfluidiser to provide stable emulsions. Droplets with a size less than 220 nm are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Preferably, substantially all (e.g. at least 90% by number) of the oil droplets have a diameter of less than 1 μm, e.g. ≦750 nm, ≦500 nm, ≦400 nm, ≦300 nm, ≦250 nm, ≦220 nm, ≦200 nm, or smaller.

One specific useful submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [19-21], as described in more detail in Chapter 10 of ref. 17 and chapter 12 of ref. 22. The MF59 emulsion advantageously includes citrate ions e.g. 10 mM sodium citrate buffer.

### C. Saponin Formulations [Chapter 22 of Ref. 17]

Saponin formulations may also be used as adjuvants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla). Gypsophilla paniculata (brides veil), and Saponaria officianalis (soup root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS 7, QS18, QS21, QH-A, QH-B and QH-C. Preferably, the saponin is QS21. A method of production of QS21 is disclosed in ref. 23. Saponin formulations may also comprise a sterol, such as cholesterol [24].

Combinations of saponins and cholesterols can be used to form unique particles called immunostimulating complexs (ISCOMs) [chapter 23 of ref. 17]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 24-26. Optionally, the ISCOMS may be devoid of additional detergent [27].

A review of the development of saponin based adjuvants can be found in refs. 28 & 29.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus. Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 30-35. Virosomes are discussed further in, for example, ref. 36

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 37. Such “small particles” of 3dMPL are small enough to be sterile filtered through a 0.22 μm membrane [37]. Other non-toxic LPS derivatives include monophosphoryl lipid A mimics, such as aminoalkyl glucosaminide phosphate derivatives e.g. RC-529 [38,39].

Lipid A derivatives include derivatives of lipid A from Escherichia coli such as OM-174. OM-174 is described for example in refs. 40 & 41.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 42, 43 and 44 disclose possible analog substitutions e.g. replacement of guanosine with 2′-deoxy-7-deazaguanosine. The adjuvant effect of CpG oligonucleotides is further discussed in refs. 45-50.

The CpG sequence may be directed to TLR9, such as the motif GTCGTT or TTCGTT [51]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 52-54. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 51 & 55-57.

A particularly useful adjuvant based around immunostimulatory oligonucleotides is known as IC31™ [58]. Thus an adjuvant used with the invention may comprise a mixture of (i) an oligonucleotide (e.g. between 15-40 nucleotides) including at least one (and preferably multiple) CpI motifs (i.e. a cytosine linked to an inosine to form a dinucleotide), and (ii) a polycationic polymer, such as an oligopeptide (e.g. between 5-20 amino acids) including at least one (and preferably multiple) Lys-Arg-Lys tripeptide sequence(s). The oligonucleotide may be a deoxynucleotide comprising 26-mer sequence 5′-(IC)₁₃-3′ (SEQ ID NO: 33). The polycationic polymer may be a peptide comprising 11-mer amino acid sequence KLKLLLLLKLK (SEQ ID NO: 34).

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 59 and as parenteral adjuvants in ref. 60. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G 192. The use of ADP-ribosylating toxins and detoxified derivatives thereof, particularly LT-K63 and LT-R72, as adjuvants can be found in refs. 61-68. A useful CT mutant is or CT-E29H [69]. Numerical reference for amino acid substitutions is preferably based on the alignments of the A and B subunits of ADP-ribosylating toxins set forth in ref. 70, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 [71], etc.) [72], interferons (e.g. interferon-γ), macrophage colony stimulating factor, and tumor necrosis factor. A preferred immunomodulator is IL-12.

### G. Bioadhesives and Mucoadhesives

Bioadhesives and mucoadhesives may also be used as adjuvants in the invention. Suitable bioadhesives include esterified hyaluronic acid microspheres [73] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [74].

### H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of ref. 17)

Examples of liposome formulations suitable for use as adjuvants are described in rets. 75-77.

### J. Polyoxyethylene Ether and Polyoxyethylene Ester Formulations

Adjuvants suitable for use in the invention include polyoxyethylene ethers and polyoxyethylene esters [78]. Such formulations further include polyoxyethylene sorbitan ester surfactants in combination with an octoxynol [79] as well as polyoxyethylene alkyl ethers or ester surfactants in combination with at least one additional non-ionic surfactant such as an octoxynol [80]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether,

### K. Polyphosphazene (PCPP)

PCPP formulations are described, for example, in refs. 81 and 82.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquamod and its homologues (e.g. “Resiquimod 3M”), described further in refs. 83 and 84.

The invention may also comprise combinations of aspects of one or more of the adjuvants identified above. For example, the following adjuvant compositions may be used in the invention: (1) a saponin and an oil-in-water emulsion [85]; (2) a saponin (e.g. QS21)+a non-toxic LPS derivative (e.g. 3dMPL) [86]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [87]; (5) combinations of 3dMPL with, for example, QS21 and/or oil-in-water emulsions [88]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 17.

The use of an aluminium hydroxide and/or aluminium phosphate adjuvant is particularly preferred, and antigens are generally adsorbed to these salts. Other preferred adjuvant combinations include combinations of Th1 and Th2 adjuvants such as CpG & alum or resiquimod & alum. A combination of aluminium phosphate and 3dMPL may be used.

### Further Antigenic Components

Compositions of the invention include modified fHBP polypeptides. It is useful if the composition should not include complex or undefined mixtures of antigens e.g. it is preferred not to include outer membrane vesicles in the composition. Polypeptides of the invention are preferably expressed recombinantly in a heterologous host and then purified.

As well as including a fHBP polypeptide, a composition of the invention may also include one or more further neisserial immunogen(s), as a vaccine which targets more than one immunogen per bacterium decreases the possibility of selecting escape mutants. Thus a composition can include a second polypeptide that, when administered to a mammal, elicits an antibody response that is bactericidal against meningococcus. The second polypeptide can be a meningococcal fHBP, but will generally not be a fHBP e.g. it may be a 287 sequence, a NadA sequence, a 953 sequence, a 936 sequence, etc.

Antigens for inclusion in the compositions include polypeptides comprising one or more of:
(a) the 446 even SEQ IDs (i.e. 2, 4, 6, . . . , 890, 892) disclosed in reference 89.
(b) the 45 even SEQ IDs (i.e. 2, 4, 6, . . . , 88, 90) disclosed in reference 90;
(c) the 1674 even SEQ IDs 2-3020, even SEQ IDs 3040-3114, and all SEQ IDs 3115-3241, disclosed in reference 3;
(d) the 2160 amino acid sequences NMB0001 to NMB2160 from reference 2;
(e) a meningococcal PorA protein, of any subtype, preferably recombinantly expressed; or
(t) a variant, homolog, ortholog, paralog, mutant etc. of (a) to (e).

Any such further neisserial immunogen may be present as a separate polypeptide to the modified fHBP of the invention of may be present as a fusion polypeptide with the modified fHBP. For instance, fusion of meningococcal 936 polypeptide and fHBP polypeptides is known [100].

A composition of the invention may include a 287 antigen. The 287 antigen was included in the published genome sequence for meningococcal serogroup B strain MC58 [91] as gene NMB2132 (GenBank accession number GI:7227388; SEQ ID NO: 10 herein). The sequences of 287 antigen from many strains have been published since then. For example, allelic forms of 287 can be seen in FIGS. 5 and 15 of reference 92, and in example 13 and FIG. 21 of reference 3 (SEQ IDs 3179 to 3184 therein). Various immunogenic fragments of the 287 antigen have also been reported. Preferred 287 antigens for use with the invention comprise an amino acid sequence: (a) having 50% or more identity (e.g. 60%, 65%, 70%, 75%. 80%, 85%, 90%, 91%, 92%, 93%, 94%, 95%, 96%, 97%, 98%, 99%, 99.5% or more) to SEQ ID NO: 10; and/or (b) comprising a fragment of at least ‘n’ consecutive amino acids of SEQ ID NO: 10, wherein ‘n’ is 7 or more (eg. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 10. The most useful 287 antigens of the invention can elicit antibodies which, after administration to a subject, can bind to a meningococcal polypeptide consisting of amino acid sequence SEQ ID NO: 10. Advantageous 287 antigens for use with the invention can elicit bactericidal anti-meningococcal antibodies after administration to a subject.

A composition of the invention may include a NadA antigen. The NadA antigen was included in the published genome sequence for meningococcal serogroup B strain MC58 [91] as gene NMB1994 (GenBank accession number GI:7227256; SEQ ID NO: 11 herein). The sequences of NadA antigen from many strains have been published since then, and the protein's activity as a Neisserial adhesin has been well documented. Various immunogenic fragments of NadA have also been reported. Preferred NadA antigens for use with the invention comprise an amino acid sequence: (a) having 50% or more identity (e.g. 60%. 65%, 70%, 75%, 80%, 85%, 90%, 91%, 92%, 93%, 94%, 95%. 96% 97%, 97%, 98%99%, 99.5% or more) to SEQ ID NO: 11; and/or (b) comprising a fragment of at least ‘n’ consecutive amino acids of SEQ ID NO: 11, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 11. The most useful NadA antigens of the invention can elicit antibodies which, after administration to a subject, can bind to a meningococcal polypeptide consisting of amino acid sequence SEQ ID NO: 11. Advantageous NadA antigens for use with the invention can elicit bactericidal anti-meningococcal antibodies after administration to a subject. SEQ ID NO: 6 is one such fragment.

A composition of the invention may include a NspA antigen. The NspA antigen was included in the published genome sequence for meningococcal serogroup B strain MC58 [91] as gene NMB0663 (GenBank accession number GI:7225888; SEQ ID NO: 12 herein). The antigen was previously known from references 93 & 94. The sequences of NspA antigen from many strains have been published since then. Various immunogenic fragments of NspA have also been reported. Preferred NspA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) comprising a fragment of at least ‘n’ consecutive amino acids of SEQ ID NO: 12, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 12. The most useful NspA antigens of the invention can elicit antibodies which, after administration to a subject, can bind to a meningococcal polypeptide consisting of amino acid sequence SEQ ID NO: 12. Advantageous NspA antigens for use with the invention can elicit bactericidal anti-meningococcal antibodies after administration to a subject.

Compositions of the invention may include a meningococcal HmbR antigen. The full-length HmbR sequence was included in the published genome sequence for meningococcal serogroup B strain MC58 [91] as gene NMB 1668 (SEQ ID NO: 13 herein). The invention can use a polypeptide that comprises a full-length HmbR sequence, but it will often use a polypeptide that comprises a partial HmbR sequence. Thus in some embodiments a HmbR sequence used according to the invention may comprise an amino acid sequence having at least i % sequence identity to SEQ ID NO: 13, where the value of i is 50, 60, 70, 80, 90, 95, 99 or more. In other embodiments a HmbR sequence used according to the invention may comprise a fragment of at least i % consecutive amino acids from SEQ ID NO: 13, where the value of j is 7, 8, 10, 12, 14, 16, 18, 20, 25, 30, 35, 40, 50, 60, 70, 80, 90, 100, 150, 200, 250 or more. In other embodiments a HmbR sequence used according to the invention may comprise an amino acid sequence (i) having at least i % sequence identity to SEQ ID NO: 13 and/or (ii) comprising a fragment of at least j consecutive amino acids from SEQ ID NO: 13. Preferred fragments of j amino acids comprise an epitope from SEQ ID NO: 13. Such epitopes will usually comprise amino acids that are located on the surface of HmbR. Useful epitopes include those with amino acids involved in HmbR's binding to haemoglobin, as antibodies that bind to these epitopes can block the ability of a bacterium to bind to host haemoglobin. The topology of HmbR, and its critical functional residues, were investigated in reference 95. The most useful HmbR antigens of the invention can elicit antibodies which, after administration to a subject, can bind to a meningococcal polypeptide consisting of amino acid sequence SEQ ID NO: 13. Advantageous HmbR antigens for use with the invention can elicit bactericidal anti-meningococcal antibodies after administration to a subject.

A composition of the invention may include a NhhA antigen. The NhhA antigen was included in the published genome sequence for meningococcal serogroup B strain MC58 [91] as gene NMB0992 (GenBank accession number GI:7226232: SEQ ID NO: 14 herein). The sequences of NhhA antigen from many strains have been published since e.g. refs 92 & 96, and various immunogenic fragments of NhhA have been reported. It is also known as Hsf. Preferred NhhA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 14; and/or (b) comprising a fragment of at least ‘n’ consecutive amino acids of SEQ ID NO: 14, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 14. The most useful NhhA antigens of the invention can elicit antibodies which, after administration to a subject, can bind to a meningococcal polypeptide consisting of amino acid sequence SEQ ID NO: 14.

Advantageous NhhA antigens for use with the invention can elicit bactericidal anti-meningococcal antibodies after administration to a subject.

A composition of the invention may include an App antigen. The App antigen was included in the published genome sequence for meningococcal serogroup B strain MC58 [91] as gene NMB1985 (GenBank accession number GI:7227246; SEQ ID NO: 15 herein). The sequences of App antigen from many strains have been published since then. Various immunogenic fragments of App have also been reported. Preferred App antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 15; and/or (b) comprising a fragment of at least ‘n’ consecutive amino acids of SEQ ID NO: 15, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 15. The most useful App antigens of the invention can elicit antibodies which, after administration to a subject, can bind to a meningococcal polypeptide consisting of amino acid sequence SEQ ID NO: 15. Advantageous App antigens for use with the invention can elicit bactericidal anti-meningococcal antibodies after administration to a subject.

A composition of the invention may include an Omp85 antigen. The Omp85 antigen was included in the published genome sequence for meningococcal serogroup B strain MC58 [91] as gene NMBO 182 (GenBank accession number GI:7225401; SEQ ID NO: 16 herein). The sequences of Omp85 antigen from many strains have been published since then. Further information on Omp85 can be found in references 97 and 98. Various immunogenic fragments of Omp85 have also been reported. Preferred Omp85 antigens for use with the invention comprise an amino acid sequence: (a) having 50% or more identity (e.g. 60%, 65%, 70%, 75%, 80%, 85%, 90%, 91%. 92%, 93%, 94%, 95%, 96%, 97%. 98%, 99%, 99.5% or more) to SEQ ID NO: 16; and/or (b) comprising a fragment of at least ‘n’ consecutive amino acids of SEQ ID NO: 16, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 16. The most useful Omp85 antigens of the invention can elicit antibodies which, after administration to a subject, can bind to a meningococcal polypeptide consisting of amino acid sequence SEQ ID NO: 16. Advantageous Omp85 antigens for use with the invention can elicit bactericidal anti-meningococcal antibodies after administration to a subject.

A composition of the invention may include a 936 antigen. The 936 antigen was included in the published genome sequence for meningococcal serogroup B strain MC58 [91] as gene NMB2091 (SEQ ID NO: 17 herein). Preferred 936 antigens for use with the invention comprise an amino acid sequence: (a) having 50% or more identity (e.g. 60%, 65%, 70%, 75%, 8, 80%, 85%, 90%, 91%, 92%, 93%, 94%, 95%, 96%, 97%, 98%%, 99%, 99.5% or more) to SEQ ID NO: 17; and/or (b) comprising a fragment of at least ‘n’ consecutive amino acids of SEQ ID NO: 17, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 17. The most useful 936 antigens of the invention can elicit antibodies which, after administration to a subject, can bind to a meningococcal polypeptide consisting of amino acid sequence SEQ ID NO: 17. The 936 antigen is a good fusion partner for fHBP (e.g. see references 99 & 100).

A composition may comprise: a polypeptide comprising SEQ ID NO: 18; a polypeptide comprising SEQ ID NO: 19; and a fusion polypeptide comprising SEQ ID NO: 17 and a fHBP of the invention (cf. refs. 99 & 100).

A composition may comprise: a polypeptide comprising SEQ ID NO: 18; a polypeptide comprising amino acids 24-350 of SEQ ID NO: 19; and a fusion polypeptide comprising SEQ ID NO: 17 and a fHBP of the invention (cf. refs. 99 & 100).

In addition to Neisserial polypeptide antigens, the composition may include antigens for immunising against other diseases or infections. For example, the composition may include one or more of the following further antigens:
a saccharide antigen from N. meningitidis serogroup A, C, W135 and/or Y, such as the saccharide disclosed in ref. 101 from serogroup C [see also ref. 102] or in ref. 103.
a saccharide antigen from Streptococcus pneumoniae [e.g. 104, 105, 106].
an antigen from hepatitis A virus, such as inactivated virus [e.g. 107, 108].
an antigen from hepatitis B virus, such as the surface and/or core antigens [e.g. 108, 109].
a diphtheria antigen, such as a diphtheria toxoid [e.g. chapter 3 of ref. 110]e.g. the CRM₁₉₇ mutant [e.g. 111].
a tetanus antigen, such as a tetanus toxoid [e.g. chapter 4 of ref. 110].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 112 & 113].
a saccharide antigen from Haemophilus influenzae B [e.g. 102].
polio antigen(s) [e.g. 114, 115] such as IPV.

measles, mumps and/or rubella antigens [e.g. chapters 9, 10 & 11 of ref. 110].
influenza antigen(s) [e.g. chapter 19 of ref. 110], such as the haemagglutinin and/or neuraminidase surface proteins.
an antigen from Moraxella catarrhalis [e.g. 116].
an protein antigen from Streptococcus agalactiae (group B streptococcus) [e.g. 117, 118].
a saccharide antigen from Streptococcus agalactiae (group B streptococcus).
an antigen from Streptococcus pyogenes (group A streptococcus) [e.g. 118, 119, 120].
an antigen from Staphylococcus aureus [e.g. 121].

The composition may comprise one or more of these further antigens.

Toxic protein antigens may be detoxified where necessary (e.g. detoxification of pertussis toxin by chemical and/or genetic means [113]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates are discussed in more detail below.

Antigens in the composition will typically be present at a concentration of at least 1 μg/ml each. In general, the concentration of any given antigen will be sufficient to elicit an immune response against that antigen.

Immunogenic compositions of the invention may be used therapeutically (i.e. to treat an existing infection) or prophylactically (i.e. to prevent future infection).

As an alternative to using proteins antigens in the immunogenic compositions of the invention, nucleic acid (preferably DNA e.g. in the form of a plasmid) encoding the antigen may be used.

In some embodiments a composition of the invention comprises in addition to the fHBP sequence, conjugated capsular saccharide antigens from 1, 2, 3 or 4 of meningococcus serogroups A, C, W135 and Y. In other embodiments a composition of the invention comprises in addition to the fl-IBP sequence, at least one conjugated pneumococcal capsular saccharide antigen.

### Meningococcus Serogroups Y, W135, C and A

Current serogroup C vaccines (Menjugate™ [122,101], Meningitec™ and NeisVac-C™) include conjugated saccharides. Menjugate™ and Meningitec™ have oligosaccharide antigens conjugated to a CRM₁₉₇ carrier, whereas NeisVac-C™ uses the complete polysaccharide (de-O-acetylated) conjugated to a tetanus toxoid carrier. The Menactra™ vaccine contains conjugated capsular saccharide antigens from each of serogroups Y, W135, C and A.

Compositions of the present invention may include capsular saccharide antigens from one or more of meningococcus serogroups Y, W135, C and A, wherein the antigens are conjugated to carrier protein(s) and/or are oligosaccharides. For example, the composition may include a capsular saccharide antigen from: serogroup C; serogroups A and C; serogroups A, C and W135; serogroups A, C and Y; serogroups C, W135 and Y; or from all four of serogroups A, C, W135 and Y.

A typical quantity of each meningococcal saccharide antigen per dose is between 1 μg and 20 μg e.g. about 1 μg, about 2.5 μg, about 4 μg, about 5 μg, or about 1 μg (expressed as saccharide).

Where a mixture comprises capsular saccharides from both serogroups A and C, the ratio (w/w) of MenA saccharide:MenC saccharide may be greater than 1 (e.g. 2:1, 3:1, 4:1, 5:1, 10:1 or higher). Where a mixture comprises capsular saccharides from serogroup Y and one or both of serogroups C and W135, the ratio (w/w) of MenY saccharide:MenW135 saccharide may be greater than 1 (e.g. 2:1, 3:1, 4:1, 5:1, 10:1 or higher) and/or that the ratio (w/w) of MenY saccharide:MenC saccharide may be less than 1 (e.g. 1:2, 1:3, 1:4, 1:5, or lower). Preferred ratios (w/w) for saccharides from serogroups A:C:W135:Y are: 1:1:1:1; 1:1:1:2; 2:1:1:1; 4:2:1:1; 8:4:2:1; 4:2:1:2; 8:4:1:2; 4:2:2:1; 2:2:1:1; 4:4:2:1; 2:2:1:2; 4:4:1:2; and 2:2:2:1. Preferred ratios (w/w) for saccharides from serogroups C:W135:Y are: 1:1:1; 1:1:2; 1:1:1; 2:1:1; 4:2:1; 2:1:2; 4:1:2; 2:2:1; and 2:1:1. Using a substantially equal mass of each saccharide is preferred.

Capsular saccharides may be used in the form of oligosaccharides. These are conveniently formed by fragmentation of purified capsular polysaccharide (e.g. by hydrolysis), which will usually be followed by purification of the fragments of the desired size.

Fragmentation of polysaccharides is preferably performed to give a final average degree of polymerisation (DP) in the oligosaccharide of less than 30 (e.g. between 10 and 20, preferably around 10 for serogroup A; between 15 and 25 for serogroups W135 and Y, preferably around 15-20; between 12 and 22 for serogroup C; etc.). DP can conveniently be measured by ion exchange chromatography or by colorimetric assays [123].

If hydrolysis is performed, the hydrolysate will generally be sized in order to remove short-length oligosaccharides [102]. This can be achieved in various ways, such as ultrafiltration followed by ion-exchange chromatography. Oligosaccharides with a degree of polymerisation of less than or equal to about 6 are preferably removed for serogroup A, and those less than around 4 are preferably removed for serogroups W135 and Y.

Preferred MenC saccharide antigens are disclosed in reference 122, as used in Menjugate™.

The saccharide antigen may be chemically modified. This is particularly useful for reducing hydrolysis for serogroup A [124; see below]. De-O-acetylation of meningococcal saccharides can be performed. For oligosaccharides, modification may take place before or after depolymerisation.

Where a composition of the invention includes a MenA saccharide antigen, the antigen is preferably a modified saccharide in which one or more of the hydroxyl groups on the native saccharide has/have been replaced by a blocking group [124]. This modification improves resistance to hydrolysis.

### Covalent Conjugation

Capsular saccharides in compositions of the invention will usually be conjugated to carrier protein(s). In general, conjugation enhances the immunogenicity of saccharides as it converts them from T-independent antigens to T-dependent antigens, thus allowing priming for immunological memory. Conjugation is particularly useful for pediatric vaccines and is a well known technique.

Typical carrier proteins are bacterial toxins, such as diphtheria or tetanus toxins, or toxoids or mutants thereof. The CRM₁₉₇ diphtheria toxin mutant [125] is useful, and is the carrier in the PREVNAR™ product. Other suitable carrier proteins include the N. meningitidis outer membrane protein complex [126], synthetic peptides [127,128], heat shock proteins [129,130], pertussis proteins [131,132], cytokines [133], lymphokines [133], hormones [133], growth factors [133], artificial proteins comprising multiple human CD4⁺ T cell epitopes from various pathogen-derived antigens [134] such as N19 [135], protein D from H. influenzae [136-138], pneumolysin [139] or its non-toxic derivatives [140], pneumococcal surface protein PspA [141], iron-uptake proteins [142], toxin A or B from C. difficile [143], recombinant P. aeruginosa exoprotein A (rEPA) [144], etc.

Any suitable conjugation reaction can be used, with any suitable linker where necessary.

The saccharide will typically be activated or functionalised prior to conjugation. Activation may involve, for example, cyanylating reagents such as CDAP (e.g. 1-cyano-4-dimethylamino pyridinium tetrafluoroborate [145,146, etc.]). Other suitable techniques use carbodiimides, hydrazides, active esters, norborane, p-nitrobenzoic acid, N-hydroxysuccinimide, S—NHS, EDC, TSTU, etc.

Linkages via a linker group may be made using any known procedure, for example, the procedures described in references 147 and 148. One type of linkage involves reductive amination of the polysaccharide, coupling the resulting amino group with one end of an adipic acid linker group, and then coupling a protein to the other end of the adipic acid linker group [149,150]. Other linkers include B-propionamido [151], nitrophenyl-ethylamine [152], haloacyl halides [153], glycosidic linkages [154], 6-aminocaproic acid [155], ADH [156], C₄ to C₁₂ moieties [157] etc. As an alternative to using a linker, direct linkage can be used. Direct linkages to the protein may comprise oxidation of the polysaccharide followed by reductive amination with the protein, as described in, for example, references 158 and 159.

A process involving the introduction of amino groups into the saccharide (e.g. by replacing terminal ═O groups with —NH_{z}) followed by derivatisation with an adipic diester (e.g. adipic acid N-hydroxysuccinimido diester) and reaction with carrier protein is preferred. Another preferred reaction uses CDAP activation with a protein D carrier e.g. for MenA or MenC.

### Outer Membrane Vesicles

It is preferred that compositions of the invention should not include complex or undefined mixtures of antigens, which are typical characteristics of OMVs. However, the invention can be used in conjunction with OMVs, as fHBP has been found to enhance their efficacy [6], in particular by over-expressing the polypeptides of the invention in the strains used for OMV preparation.

This approach may be used in general to improve preparations of N. meningitidis serogroup B microvesicles [160], ‘native OMVs’ [161], blebs or outer membrane vesicles [e.g. refs. 162 to 167, etc.]. These may be prepared from bacteria which have been genetically manipulated [168-171] e.g. to increase immunogenicity (e.g. hyper-express immunogens), to reduce toxicity, to inhibit capsular polysaccharide synthesis, to down-regulate PorA expression, etc. They may be prepared from hyperblebbing strains [172-175]. Vesicles from a non-pathogenic Neisseria may be included [176]. OMVs may be prepared without the use of detergents [177,178]. They may express non-Neisserial proteins on their surface [179]. They may be LPS-depleted. They may be mixed with recombinant antigens [162,180]. Vesicles from bacteria with different class I outer membrane protein subtypes may be used e.g. six different subtypes [181,182] using two different genetically-engineered vesicle populations each displaying three subtypes, or nine different subtypes using three different genetically-engineered vesicle populations each displaying three subtypes, etc. Useful subtypes include: P1.7,16; P1.5-1, 2-2; P1,19,15-1; P1.5-2,10; P1.12-1,13; P1.7-2,4; P1.22,14; P1.7-1,1; P1.18-1,3,6.

Further details are given below.

### Protein Expression

Bacterial expression techniques are known in the art. A bacterial promoter is any DNA sequence capable of binding bacterial RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in .Escherichia coli (E. coli) [Raibaud et al. (1984) Annu. Rev. Genes. 18:173]. Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) [Chang et al. (1977) Nature 198:1056], and maltose. Additional examples include promoter sequences derived from biosynthetic enzymes such as tryptophan (trp) [Goeddel et al. (1980) Nuc. Acids Res. 8:4057; Yelverton et al. (1981) Nucl. Acids Res. 9:731; U.S. Pat. No. 4,738,921; EP-A-0036776 and EP-A-0121775]. The β-lactamase (bla) promoter system [Weissmann (1981) “The cloning of interferon and other mistakes.” In Inteferon 3 (ed. I. Gresser)], bacteriophage lambda PL [Shimatake et al. (1981) Nature 292:128] and T5 [U.S. Pat. No. 4,689,406] promoter systems also provide useful promoter sequences. Another promoter of interest is an inducible arabinose promoter (pBAD).

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433]. For example, the ace promoter is a hybrid trp-lac promoter comprised of both trp promoter and lac operon sequences that is regulated by the lac repressor [Amann et al. (1983) Gene 25:167; de Boer et al. (1983) Proc. Natl. Acad. Sci. 80:21]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al. (1986) J. Mol. Biol. 189:113; Tabor et al. (1985) Proc Natl. Acad. Sci. 82:1074]. In addition, a hybrid promoter can also be comprised of a bacteriophage promoter and an E. coli operator region (EP-A-0 267 851).

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of the initiation codon. The SD sequence is thought to promote binding of mRNA to the ribosome by the pairing of bases between the SD sequence and the 3′ and of E. coli 16S rRNA [Steitz et al. (1979) “Genetic signals and nucleotide sequences in messenger RNA,” In Biological Regulation and Development: Gene Expression (ed. R. F. Goldberger)]. To express cukaryotic genes and prokaryotic genes with weak ribosome-binding site [Sambrook et al. (1989) “Expression of cloned genes in Escherichia coli.” In Molecular Cloning: A Laboratory Manual].

A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo on in vitro incubation with a bacterial methionine N-terminal peptidase (EP-A-0219237).

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (e.g. plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome (EP-A-0127328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline [Davies et al. (1978) Annu. Rev. Microbiol. 32:469]. Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis [Palva et al. (1982) Proc. Natl. Acad. Sci. USA 79:5582; EP-A-0036259 and EP-A-0063953; WO84/04541, Escherichia coli [Shimatake et al. (1981) Nature 292:128; Amann et al. (1985) Gene 40:183; Studier et al. (1986) J. Mol. Biol. 189:113; EP-A-0 036 776, EP-A-0 136 829 and EP-A-0 136 907], Streptococcus cremoris [Powell et al. (1988) Appl. Environ. Microbiol. 54:655]; Streptococcus lividans [Powell et al. (1988) Appl. Environ. Microbiol. 54:655], Streptomyces lividans [U.S. Pat. No. 4,745,056].

Methods of introducing exogenous DNA into bacterial hosts are well-known in the art, and usually include either the transformation of bacteria treated with CaCl₂ or other agents, such us divalent cations and DMSO. DNA can also be introduced into bacterial cells by electroporation. Transformation procedures usually vary with the bacterial species to be transformed. See e.g. [Masson et al. (1989) FEMS Microbial. Lett. 60:273; Palva et al. (1982) Proc. Natl. Acad. Sci. USA 79:5582; EP-A-0036259 and EP-A-0063953; WO84/04541, Bacillus], [Miller et al. (1988) Proc. Natl. Acad. Sci. 85:856; Wang et al. (1990) J. Bacterial. 172:949, Campylobacter], [Cohen et al. (1973) Proc. Natl. Acad. Sci. 69:2110; Dower et al. (1988) Nucleic Acid Res. 16:6127; Kushner (1978) “An improved method for transformation of Escherichia coli with ColE1-derived plasmids. In Genetic Engineering Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; Escherichia], [Chassy et al. (1987) FEMS Microbiol. Lett. 44:173 Lactobacillus]; [Fiedler et al. (1988) Anal. Biochem 170:38, Pseudomonas]; [Augustin et al. (1990) FEMS Microbiol. Lett. 66:203, Staphylococcus], [Barany et al. (1980) J. Bacteriol. 144:698; Harlander (1987) “Transformation of Streptococcus lactis by electroporation, in: Streptococcal Genetics (ed. J. Ferretti and R. Curtiss III); Perry et al. (1981) Infect. Inmmun. 32:1295; Powell et al. (1988) Appl. Environ. Microbiol. 54:655; Somkuti et al. (1987) Proc. 4th Evr. Cong. Biotechnology 1:412, Streptococcus].

### Host Cells

The invention provides a bacterium which expresses a polypeptide of the invention. The bacterium may be a meningococcus. The bacterium may constitutively express the polypeptide, but in some embodiments expression may be under the control of an inducible promoter. The bacterium may hyper-express the polypeptide (cf. ref. 183). Expression of the polypeptide may not be phase variable.

The invention also provides outer membrane vesicles prepared from a bacterium of the invention. It also provides a process for producing vesicles from a bacterium of the invention. Vesicles prepared from these strains preferably include the polypeptide of the invention, which should be in an immunoaccessible form in the vesicles i.e. an antibody which can bind to purified polypeptide of the invention should also be able to bind to the polypeptide which is present in the vesicles.

These outer membrane vesicles include any proteoliposomic vesicle obtained by disruption of or blebbling from a meningococcal outer membrane to form vesicles therefrom that include protein components of the outer membrane. Thus the term includes OMVs (sometimes referred to as ‘blebs’), microvesicles (MVs [160]) and ‘native OMVs’ (‘NOMVs’ [161]).

MVs and NOMVs are naturally-occurring membrane vesicles that form spontaneously during bacterial growth and are released into culture medium. MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 174 & 175 describe Neisseria with high MV production.

OMVs are prepared artificially from bacteria, and may be prepared using detergent treatment (e.g. with deoxycholate), or by non-detergent means (e.g. see reference 178). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [184 & 185] being preferred for treating Neisseria) at a pH sufficiently high not to precipitate the detergent [186]. Other techniques may be performed substantially in the absence of detergent [178] using techniques such as sonication, homogenisation, microfluidisation, cavitation, osmotic shock, grinding, French press, blending, etk. Methods using no or low detergent can retain useful antigens such as NspA [178]. Thus a method may use an OMV extraction buffer with about 0.5% deoxycholate or lower e.g. about 0.2%, about 0.1%, <0.05% or zero.

A useful process for OMV preparation is described in reference 187 and involves ultrafiltration on crude OMVs, rather than instead of high speed centrifugation. The process may involve a step of ultracentrifugation after the ultrafiltration takes place.

Vesicles for use with the invention can be prepared from any meningococcal strain. The vesicles will usually be from a serogroup B strain, but it is possible to prepare them from serogroups other than B (e.g. reference 186 discloses a process for serogroup A), such as A, C, W135 or Y. The strain may be of any serotype (e.g. 1, 2a, 2b, 4, 14, 15, 16, etc.), any serosubtype, and any immunotype (e.g. L1; L2; L3; L3,3,7; L10; etc.). The meningococci may be from any suitable lineage, including hyperinvasive and hypervirulent lineages e.g. any of the following seven hypervirulent lineages: subgroup I; subgroup III; subgroup IV-I; ET-5 complex; ET-37 complex; A4 cluster; lineage 3.

Bacteria of the invention may, in addition to encoding a polypeptide of the invention, have one or more further modifications. For instance, they may have a modified fur gene [188]. Expression of nspA expression may be up-regulated with concomitant porA and cps knockout. Further knockout mutants of N. meningitidis for OMV production are disclosed e.g. in reference 193. Reference 189 discloses the construction of vesicles from strains modified to express six different PorA subtypes. Mutant Neisseria with low endotoxin levels, achieved by knockout of enzymes involved in LPS biosynthesis, may also be used [190,191]. These or others mutants can all be used with the invention.

Thus a strain used with the invention may in some embodiments express more than one PorA subtype. 6-valent and 9-valent PorA strains have previously been constructed. The strain may express 2, 3, 4, 5, 6, 7, 8 or 9 of PorA subtypes: P1.7,16; P1.5-1, 2-2; P1,19,15-1; P1.5-2,10; P1.12-1,13; P1.7-2,4; P1.22,14: P1.7-1,1 and/or P1.18-1,3,6. In other embodiments a strain may have been down-regulated for PorA expression e.g. in which the amount of PorA has been reduced by at least 20% (e.g. ≧30%, ≧40%, ≧50%, ≧60%, ≧70%, ≧80%, ≧90%, ≧95%, etc.), or even knocked out, relative to wild-type levels (e.g. relative to strain H44/76).

In some embodiments a strain may hyper-express (relative to the corresponding wild-type strain) certain proteins. For instance, strains may hyper-express NspA, protein 287 [162], fHBP [183], TbpA and/or TbpB [180], CuZn-superoxide dismutase, HmbR, etc.

A gene encoding a polypeptide of the invention may be integrated into the bacterial chromosome or may be present in episomal form e.g. within a plasmid.

Advantageously for vesicle production, a meningococcus may be genetically engineered to ensure that expression of the polypeptide is not subject to phase variation. Methods for reducing or eliminating phase variability of gene expression in meningococcus are disclosed in reference 192. For example, a gene may be placed under the control of a constitutive or inducible promoter, or by removing or replacing the DNA motif which is responsible for its phase variability.

In some embodiments a strain may include one or more of the knockout and/or hyper-expression mutations disclosed in references 166, 168, 172, and 193. Preferred genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB; (b) CtrA, CtrB, CtrC, CtrD, FrpB, GalE, HtrB/MsbB, LbpA, LbpB. LpxK, Opa, Opc, PhoP, PilC, PmrE, PmrF, SiaA, SiaB, SiaC, SiaD, TbpA, and/or TbpB; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB; and (d) CtrA, CtrB, CtrD, FrpB, OpA, OpC, PilC, PorB, SiaD, SynA, SynB, and/or SynC.

Where a mutant strain is used, in some embodiments it may have one or more, or all, of the following characteristics: (i) down-regulated or knocked-out LgtB and/or GalE to truncate the meningococcal LOS; (ii) up-regulated TbpA; (iii) up-regulated NhhA; (iv) up-regulated Omp85; (v) up-regulated LbpA; (vi) up-regulated NspA; (vii) knocked-out PorA; (viii) down-regulated or knocked-out FrpB; (ix) down-regulated or knocked-out Opa; (x) down-regulated or knocked-out Opc; (xii) deleted cps gene complex. A truncated LOS can be one that does not include a sialyl-lacto-N-neotetraose epitope e.g. it might be a galactose-deficient LOS. The LOS may have no a chain.

Depending on the meningococcal strain used for preparing the vesicles, they may or may not include the strain's native fHBP antigen [194].

If LOS is present in a vesicle it is possible to treat the vesicle so as to link its LOS and protein components (“intra-bleb” conjugation [193]).

### General

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

“Sequence identity” is preferably determined by the Smith-Waterman homology search algorithm as implemented in the MPSRCH program (Oxford Molecular), using an affine gap search with parameters gap open penalty=12 and gap extension penalty=1.

After serogroup, meningococcal classification includes serotype, serosubtype and then immunotype, and the standard nomenclature lists serogroup, serotype, serosubtype, and immunotype, each separated by a colon e.g. B:4:P1.15:L3,7,9. Within serogroup B, some lineages cause disease often (hyperinvasive), some lineages cause more severe forms of disease than others (hypervirulent), and others rarely cause disease at all. Seven hypervirulent lineages are recognised, namely subgroups 1, III and IV-1, ET-5 complex, ET-37 complex, A4 cluster and lineage 3. These have been defined by multilocus enzyme electrophoresis (MLEE), but multilocus sequence typing (MLST) has also been used to classify meningococci. The four main hypervirulent clusters are ST32, ST44, ST8 and ST11 complexes.

In general, the invention does not encompass the various fHBP sequences specifically disclosed in references 4, 5, 7, 8, 9, 195, 196, 197, 198, 199, 200 and 201.

### MODES FOR CARRYING OUT THE INVENTION

### fHBP Mutations

Reference 10 discloses a mutant fHBP referred to as ‘E283A,E304A’ in which glutamate residues at positions 237 and 258 of SEQ ID NO: 1 were mutated to alanine. Surface plasmon resonance showed that the affinity of the double mutant protein was reduced by more than two orders of magnitude relative to the unmutated protein, with almost no detectable interaction when reagents were used at 50 nM. The authors did not report on any immunogenicity of the mutant protein.

FACS was used to study binding of human fH to live meningococci. The assay confirmed that fH binds to bacteria in all test strains. Dose-related binding was evident. Incubation with polyclonal anti-fHBP (1:100 ratio) could inhibit the binding.

Mutants strains were made in which the natural fHBP gene was replaced with the double glutamate mutant. FACS confirmed ref. 10's finding that these mutant strains did not appreciably bind fH. Binding of fH was similar in the mutant strain and in a ΔfHBP knockout strain. In contrast, anti-fHBP serum bound to the wild-type strains and the mutant strains, but not the ΔfhBP strain.

Sera obtained from human patients immunised with the vaccine disclosed in reference 100 were tested by SBA assay for bactericidal efficacy against recombinant strains. There was no significant difference in SBA sensitivity between a recombinant strain having (i) a wild-type fHBP or (ii) the mutant fHBP. These data suggest that fH binding does not affect bactericidal activity.

Thus fHBP's ability to bind to fH can be uncoupled from its immunogenicity. This finding means that fHBP can be improved as an antigen. The protein can be engineered to minimise its interactions with fH while retaining its immunogenic properties. Reduced fH binding means, for instance, that the protein's epitopes will not be obscured in the body by fH e.g. the protein can be optimised for presentation to the immune system without interference by fH.

### NMR Study

Reference 10 used X-ray crystallography to study the interaction between fHBP and complement control protein (CCP) domains 6 and 7 of fH. In contrast, NMR has been used to study the solution interactions between fHBP and CCP domains 5 to 7. HSQC was used to analyse ¹⁵N-labelled fHBP with or without CCP domains 5 to 7 of human fH (molecular ratio 1:1). These experiments identified residues which interact with fH or whose conformation changes due to that interaction.

Residues 37, 38, 41, 42, 43, 45, 56, 80, 82, 83, 84, 86, 89, 91, 95, 112, 115, 116, 119, 121, 122, 124, 126, 127, 128, 129, 130, 139, 141, 143, 160, 163, 188, 198, 199, 207, 210, 211, 213, 219, 220, 221, 223, 237, 241, 242 and 248 (numbered according to SEQ ID NO: 4) are surface-exposed residues which were perturbed by the fHBP interaction. Residues 31, 32, 36, 39, 40, 44, 57, 64, 74, 76, 78, 80, 93, 96, 97, 98, 99, 101, 103, 107, 109, 110, 111, 129, 132, 135, 152, 165, 177, 179, 196, 198, 206, 212, 224, 225, 226, 236, 238, 248, 249, 250 and 251 were also perturbed but are buried.

These residues define an extensive region which involves both N- and C-terminal domains of fHBP. Notably, surface-exposed residues located in the linker connecting N- and C-domains of fHBP (Thr139, Phe141, Asp142 and Lys143) and several buried residues located at the domain-domain interface of fHBP (Gln97, Tyr99. Gln101, His103, Phe129, Gly132, Ala135, Ile226, Gly236, Ser237, His 248, Ile249, Gly250 and Leu251) were perturbed, suggesting that a molecular rearrangement of fHbp occurs during the formation of the complex.

The total number of perturbed surface-exposed residues in solution define a larger contact area than seen in reference 10, but still contains all the residues seen therein. Two important exceptions are represented by Glu218 and Glu239, which seem to be marginally affected in the NMR experiment.

Discrepancies can be explained assuming that a conformational changes occurs in the molecule. The higher number of perturbed residues can be justified by a model of interaction for fHBP-fH complex in which the reciprocal orientation of fHBP's N- and C-domains changed if compared with the structure of the free fHBP. Other differences could be ascribed to additional contact between fHbp and fH domain 7

Mutant fHBP Sequences

The NMR structure provides residues which can be mutated in fHBP to reduce the protein's interactions with fH. Residues can be mutated individually or in combination, and the resulting protein can be tested using routine assays (i) for fH interaction and (ii) ability to elicit bactericidal antibodies. For instance, the following residues in the MC58 antigen are mutated to alanine and then tested: 43, 45, 56, 83, 112, 116, 119, 122, 127, 139, 141, 142, 143, 198, 211, 219, 221, 241. Thus, for example, the methods provide proteins comprising SEQ ID NOs: 23 to 27.

These residues are arranged into four clusters, A to D:
A: residues 112, 116, 119, 122, 127.
B: residues 43, 45, 56, 83.
C: residues 211, 219, 221, 241.
D: residues 139, 141, 142, 143, 198.

Each cluster mainly consists of residues identified by the NMR experiments, and each defines a distinct region on the protein surface.

Preliminary experiments showed that mutations in cluster A residues affected fH/fHBP binding.

The identified residues are suitable not only for modification in wild-type sequences. For instance, reference 201 discloses forms of fHBP which have been modified to increase their ability to elicit inter-family anti-fHBP bactericidal antibodies (e.g. SEQ ID NOs: 20 to 22 herein). These proteins can be further modified at the NMR-identified residues to decrease their fH-binding activity while retaining their useful immunogenic properties. For example, SEQ ID NO: 20 includes Asp-37 from SEQ ID NO: 4 (Asp-30 by SEQ ID NO: 20's own numbering). This residue can be mutated (e.g. to glycine, to provide SEQ ID NO: 28) and (i) the affinity of its interaction with fH can be tested using the methods of ref. 10, and (ii) its ability to elicit bactericidal antibodies can be tested using the methods of ref. 4.

### Siderophore Binding

The fHBP includes a β-barrel domain with strong structural homology to lipocalin. Meningococcal fHBP was mixed with four different iron-loaded siderophores (enterobactin, salmochelin, yersiniabactin, aerobactin) and digested with trypsin. The digestion pattern was similar to the control for all samples except for the mixtures with enterobactin and salmochelin, where a trace of undigested protein remained. Size-exclusion chromatography showed a co-elution of fHBP and enterobactin, but this co-elution was not seen with a negative control. Native PAGE also indicated an interaction between fHBP and enterobactin.

A BC fragment of fHBP, containing the β-barrel, was also able to interact with enterobactin.

After 24 hours of incubation with enterobactin or salmochelin, high MW bands were visible by SDS-PAGE, indicating that the siderophores were mediating fHBP dimerisation (or trimerisation).

NMR studies revealed residues whose signal was perturbed in the presence of enterobactin. Numbered according to SEQ ID NO: 4, residues were 102, 136-138, 148-154, 166, 205, 230 and 254. These residues are all located in a well defined area, indicating a specific interaction. Unlike siderocalin, which binds enterobactin inside its β-barrel, fHBP interacts on the barrel's outer surface. In particular, Arg and Lys residues are involved (Arg-149, Arg-153, Lys-230, Lys-254).

The residues which interact with enterobactin are different from the residues which interact with fH. Thus fHBP might bind simultaneously to fH and to a siderophore.

Biacore assays using immobilised fHBP also confirmed an interaction with iron-loaded enterobactin. The enterobactin binds to the fHBP in a dose-dependent manner with micromolar affinity. Binding to salmochelin (another catecholate) was also seen, but not to yersiniabactin or aerobactin.

fHBP was tested in a serum bactericidal assay both with and without pre-incubation with enterobactin. The presence of enterobactin had no impact on bactericidal titres.

To eliminate the siderophore interaction the amino acid residues 102, 136-138, 148-154, 230 and/or 254 can be mutated. This numbering is according to SEQ ID NO: 4 and the corresponding amino acid residues in SEQ ID NOs: 5 and 6 can easily be identified by alignment. Using SEQ ID NO: 4 as a starting point, for instance residues Arg-149, Tyr-152, Arg-153, and/or Lys-254 can be substituted with alanine to provide SEQ ID NOs: 29-32.

It will be understood that the invention is described above by way of example only and modifications may be made whilst remaining within the scope and spirit of the invention.

### REFERENCES

[1] Jodar et al. (2002) Lancet 359(9316): 1499-1508.
[2] Pizza et a. (2000) Science 287:1816-1820.
[3] WO99/57280.
[4] Masignani et al. (2003) J Exp Med 197:789-799.
[5] Welsch et al. (2004) J Immunol 172:5605-15.
[6] Hou et al. (2005) J Infect Dis 192(4):580-90.
[7] WO03/063766.
[8] Fletcher et a. (2004) Infect Immun 72:2088-2100.
[9] Zhu et al. (2005) Infect Immun 73(10):6838-45.
[10] Schneider et al. (2009) Nature 458:890-5.
[11] WO2010/046715.
[12] Needleman & Wunsch (1970) J. Mol. Biol. 48, 443-453.
[13] Rice et al. (2000) Trends Genet. 16:276-277.
[14] WO01/30390.
[15] Gennaro (2000) Remington: The Science and Practice of Pharmacy. 20th edition. ISBN: 0683306472.
[16] WO03/009869.
[17] Vaccine Design . . . (1995) eds. Powell & Newman. ISBN: 030644867X. Plenum.
[18] WO00/23105.
[19] WO90/14837.
[20] Podda & Dcl Giudice (2003) Expert Rev Vaccines 2:197-203.
[21] Podda (2001) Vaccine 19: 2673-2680.
[22] Vaccine Adjuvants: Preparation Methods and Research Protocols (Volume 42 of Methods in Molecular Medicine series). ISBN: 1-59259-083-7. Ed. O'Hagan.
[23] U.S. Pat. No. 5,057,540.
[24] WO96/33739.
[25] EP-A-0109942.
[26] WO96/11711.
[27] WO00/07621.
[28] Barr et al. (1998) Advanced Drug Delivery Reviews 32:247-271.
[29] Sjolanderet et al. (1998) Advanced Drug Delivery Reviews 32:321-338.
[30] Niikura et al. (2002) Virology 293:273-280.
[31] Lenz et al. (2001) J Immunol 166:5346-5355.
[32] Pinto et al. (2003) J Infect Dis 188:327-338.
[33] Gerber et al. (2001) J Virol 75:4752-4760.
[34] WO03/024480.
[35] WO03/024481.
[36] Gluck et al. (2002) Vaccine 20:B10-B16.
[37] EP-A-0689454.
[38] Johnson et al. (1999) Bioorg Med Chem Lett 9:2273-2278.
[39] Evans et al. (2003) Expert Rev Vaccines 2:219-229.
[40] Meraldi et al. (2003) Vaccine 21:2485-2491.
[41] Pajak et al. (2003) Vaccine 21:836-842.
[42] Kandimalla et al. (2003) Nucleic Acids Research 31:2393-2400.
[43] WO02/26757.
[44] WO99/62923.
[45] Krieg (2003) Nature Medicine 9:831-835.
[46] McCluskie et al. (2002) FEMS Immunology and Medical Microbiology 32:179-185.
[47] WO98/40100.
[48] U.S. Pat. No. 6,207,646.
[49] U.S. Pat. No. 6,239,116.
[50] U.S. Pat. No. 6,429,199.
[51] Kandimalla et al. (2003) Biochemical Society Transactions 31 (part 3):654-658.
[52] Blackwell et al. (2003) J Immunol 170:4061-4068.
[53] Krieg (2002) Trends Immunol 23:64-65.
[54] WO01/95935.
[55] Kandimalla et al. (2003) BBRC 306:948-953.
[56] Bhagat et al. (2003) BBRC 300:853-861.
[57] WO03/035836.
[58] Schellack et al. (2006) Vaccine 24:5461-72.
[59] WO95/17211.
[60] WO98/42375.
[61] Beignon et al. (2002) Inject Immun 70:3012-3019.
[62] Pizza et al. (2001) Vaccine 19:2534-2541.
[63] Pizza et at. (2000) Int J Med Microbiol 290:455-461.
[64] Scharton-Kersten et al. (2000) Infect Immun 68:5306-5313.
[65] Ryan et al. (1999) Infect Immun 67:6270-6280.
[66] Partidos et al. (1999) Immunol Lett 67:209-216.
[67] Peppoloni et al. (2003) Expert Rev Vaccines 2:285-293.
[68] Pine et al. (2002) J Control Release 85:263-270.
[69] Tebbey et al. (2000) Vaccine 18:2723-34.
[70] Domenighini et al. (1995) Mol Microbiol 15:1165-1167.
[71] WO99/40936.
[72] WO99/44636.
[73] Singh et al] (2001) J Cont Release 70:267-276.
[74] WO99/27960.
[75] U.S. Pat. No. 6,090,406.
[76] U.S. Pat. No. 5,916,588.
[77] EP-A-0626169.
[78] WO99/52549.
[79] WO01/21207.
[80] WO01/21152.
[81] Andrianov et al. (1998) Biomaterials 19:109-115.
[82] Payne et al. (1998) Adv Drug Delivery Review 31:185-196.
[83] Stanley (2002) Clin Exp Dermatol 27:571-577.
[84] Jones (2003) Curr Opin Investig Drugs 4:214-218.
[85] WO99/11241.
[86] WO94/00153.
[87] WO98/57659.
[88] European patent applications 0835318, 0735898 and 0761231.
[89] WO99/24578.
[90] WO99/36544.
[91] Tettelin et al. (2000) Science 287:1809-1815.
[92] WO00/66741.
[93] Martin et al. (1997) J Exp Med 185(7):1173-83.
[94] WO96/29412.
[95] Perkin-Balding et al. (2003) Microbiology 149:3423-35.
[96] WO01/55182.
[97] WO01/38350.
[98] WO00/23595.
[99] WO2004/032958.
[100] Giuliani et al. (2006) Proc Natl Acad Sci USA. 103:10834-9.
[101] Costantino et al. (1992) Vaccine 10:691-698.
[102] Costantino et al. (1999) Vaccine 17:1251-1263.
[103] WO03/007985.
[104] Watson (2000) Pediatr Infect Dis J 19:331-332.
[105] Rubin (2000) Pediatr Clin North Am 47:269-285, v.
[106] Jedrzejas (2001) Microbiol Mol Biol Rev 65:187-207.
[107] Bell (2000) Pediatr Infect Dis J 19:1187-1188.
[108] Iwarson (1995) APMIS 103:321-326.
[109] Gerlich et al. (1990) Vaccine 8 Suppl:S63-68 & 79-80.
[110] Vaccines (1988) eds. Plotkin & Mortimer. ISBN 0-7216-1946-0.
[111] Del Guidice et al. (1998) Molecular Aspects of Medicine 19:1-70.
[112] Gustafsson et al. (1996) N. Engl. J. Med. 334:349-355.
[113] Rappuoli et al. (1991) TIBTECH 9:232-238.
[114] Sutter et al. (2000) Pediatr Clin North Am 47:287-308.
[115] Zimmerman & Spann (1999) Am Fam Physician 59:113-118, 125-126.
[116] McMichael (2000) Vaccine 19 Suppl 1:S101-107.
[117] Schuchat (1999) Lancet 353(9146):51-6.
[118] WO02/34771.
[119] Dale (1999) Infect Dis Clin North Am 13:227-43, viii.
[120] Ferretti et al. (2001) PNAS USA 98: 4658-4663.
[121] Kuroda et al. (2001) Lancet 357(9264):1225-1240; see also pages 1218-1219.
[122] Jones (2001) Curr Opin Investig Drugs 2:47-49.
[123] Ravenscroft et al. (1999) Vaccine 17:2802-2816.
[124] WO03/080678.
[125] Research Disclosure, 453077 (January 2002).
[126] EP-A-0372501.
[127] EP-A-0378881.
[128] EP-A-0427347.
[129] WO93/17712.
[130] WO94/03208.
[131] WO98/58668.
[132] EP-A-0471177.
[133] WO91/01146.
[134] Falugi et al. (2001) Eur J Immunol 31:3816-3824.
[135] Baraldo et al. (2004) Infect Immun 72(8):4884-7.
[136] EP-A-0594610.
[137] Ruan et al., (1990) J Immunol 145:3379-3384.
[138] WO00/56360.
[139] Kuo et al. (1995) Infect Immun 63:2706-13.
[140] Michon et al. (1998) Vaccine. 16:1732-41.
[141] WO02/091998.
[142] WO01/72337.
[143] WO0/61761.
[144] WO00/33882
[145] Lees et a. (1996) Vaccine 14:190-198.
[146] WO95/08348.
[147] U.S. Pat. No. 4,882,317
[148] U.S. Pat. No. 4,695,624
[149] Porro et al. (1985) Mol Immunol 22:907-919.s
[150] EP-A-0208375
[151] WO00/10599
[152] Gever et al. Med. Microbiol. Immunol, 165: 171-288 (1979).
[153] U.S. Pat. No. 4,057,685.
[154] U.S. Pat. Nos. 4,673,574; 4,761,283; 4,808,700.
[155] U.S. Pat. No. 4,459,286.
[156] U.S. Pat. No. 4,965,338
[157] U.S. Pat. No. 4,663,160.
[158] U.S. Pat. No. 4,761,283
[159] U.S. Pat. No. 4,356,170
[160] WO02/09643.
[161] Katial et al. (2002) Infect Immun 70:702-707.
[162] WO01/52885.
[163] European patent 0301992.
[164] Bjune et al. (1991) Lancet 338(8775):1093-1096.
[165] Fukasawa et al. (1999) Vaccine 17:2951-2958.
[166] WO02/09746.
[167] Rosenqvist et al. (1998) Dev. Biol. Stand. 92:323-333.
[168] WO01/09350.
[169] European patent 0449958.
[170] EP-A-0996712.
[171] EP-A-0680512.
[172] WO02/062378.
[173] WO99/59625.
[174] U.S. Pat. No. 6,180,111.
[175] WO0134642.
[176] WO03/051379.
[177] U.S. Pat. No. 6,558,677.
[178] WO2004/019977.
[179] WO02/062380.
[180] WO0/25811.
[181] Peeters et al. (1996) Vaccine 14:1008-1015.
[182] Vermont et al. (2003) Infect Inmmun 71:1650-1655.
[183] WO2006/081259.
[184] European patent 0011243.
[185] Fredriksen et al. (1991) NIPH Ann. 14(2):67-80.
[186] WO01/91788.
[187] WO2005/004908.
[188] WO98/56901.
[189] Claasasen et al. (1996) 14(10):1001-8.
[190] WO99/10497.
[191] Steeghs et al. (2001) The EMBO Journal 20:6937-6945.
[192] WO2004/015099.
[193] WO2004/014417.
[194] WO2004/046177.
[195] WO01/64920.
[196] WO03/020756.
[197] WO2004/048404.
[198] WO2004/094596
[199] WO2006/024954.
[200] WO2007/060548.
[201] WO2009/104097.

1: A polypeptide comprising an amino acid sequence: (a) which has at least 85% identity to any one of SEQ ID NOs: 4, 5 or 6, and/or comprises a fragment of SEQ ID NO: 4, 5 or 6; but (b) wherein one or more of the following amino acid residues from SEQ ID NO: 4, 5 or 6 is either absent or is substituted by a different amino acid:

| SEQ ID NO: 4 | SEQ ID NO: 5 | SEQ ID NO: 6 |
|---|---|---|
| Asp-37 | Asp-37 | Glu-42 |
| Lys-45 | Lys-45 | Thr-50 |
| Thr-56 | Thr-56 | Thr-61 |
| Glu-83 | Glu-83 | Glu-91 |
| Glu-95 | Glu-95 | Glu-103 |
| Glu-112 | Glu-112 | Glu-120 |
| Lys-122 | Ser-122 | Ser-130 |
| Val-124 | Ile-124 | Ile-132 |
| Arg-127 | Arg-127 | Arg-135 |
| Thr-139 | Thr-139 | Thr-147 |
| Phe-141 | Phe-141 | Phe-149 |
| Asp-142 | Asn-142 | Asn-150 |
| Lys-143 | Gln-143 | Gln-151 |
| Ile-198 | Leu-197 | Leu-205 |
| Ser-211 | Asp-210 | Asp-218 |
| Leu-213 | Arg-212 | Arg-220 |
| Lys-219 | Lys-218 | Lys-226 |
| Asn-43 | Asn-43 | Asn-48 |
| Asp-116 | Asn-116 | Asn-124 |
| His-119 | Lys-119 | Lys-127 |
| Ser-221 | Thr-220 | Thr-228 |
| Lys-241 | Lys-240 | Lys-248 |

wherein the polypeptide (i) can, after administration to a host animal, elicit antibodies which can recognise a wild-type meningococcal polypeptide consisting of SEQ ID NO: 4, 5 or 6, and (ii) has a lower affinity for human factor H than the same polypeptide but without the modification(s) of (b).

2: The polypeptide of claim 1, comprising an amino acid sequence which has at least 85% identity to SEQ ID NO: 4 and/or comprises a fragment of SEQ ID NO: 4, and which can, after administration to a host animal, elicit antibodies which can recognise a wild-type meningococcal polypeptide consisting of SEQ ID NO: 4.

3: A method for designing a modified fHBP amino acid sequence comprising steps of: (i) providing a starting amino acid sequence, wherein a protein consisting of or comprising the starting amino acid sequence can bind to human factor H; (ii) identifying within the starting amino acid sequence an amino acid residue which, using a pairwise alignment algorithm, aligns with a residue in SEQ ID NO: 4, 5 or 6 as listed in the table in claim 1; (iii) either deleting the amino acid identified in step (ii), or replacing it with a different amino acid, thereby providing the modified fHBP amino acid sequence.

4: A polypeptide comprising (i) a modified fHBP amino acid sequence designed by the method of claim 3, or (ii) an amino acid sequence selected from SEQ ID NOs: 23 to 32.

5: Nucleic acid encoding the polypeptide of claim 1.

6: A plasmid comprising a nucleotide sequence encoding the polypeptide of claim 1.

7: A host cell transformed with the plasmid of claim 6.

8: The host cell of claim 7, wherein the cell is a meningococcal bacterium.

9: Membrane vesicles, wherein the vesicles include a polypeptide of claim 1.

10: An immunogenic composition comprising a polypeptide of claim 1.

11: The composition of claim 10, including an adjuvant.

12: The composition of claim 11, wherein the adjuvant comprises an aluminium salt.

13: The composition of claim 10, further comprising a second polypeptide that, when administered to a mammal, elicits an antibody response that is bactericidal against meningococcus, provided that the second polypeptide is not a meningococcal fHBP.

14: The composition of claim 10, further comprising a conjugated capsular saccharide from N. meningitidis serogroup A, C, W135 and/or Y.

15: The composition of claim 10, further comprising a conjugated pneumococcal capsular saccharide.

16: A method for raising an antibody response in a mammal, comprising administering an immunogenic composition of claim 10.


SEQUENCE LISTING (35 sequences)

SEQ ID NO: 1 - protein, 274 aa, Neisseria meningitidis
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 281 aa, Neisseria meningitidis
MNRTAFCCLSLTTALILTACSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 5 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 6 - protein, 262 aa, Neisseria meningitidis
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 7 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 8 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 9 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 10 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 11 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 12 - protein, 174 aa, Neisseria meningitidis
MKKALATLIALALPAAALAEGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSAGVRVKF

SEQ ID NO: 13 - protein, 792 aa, Neisseria meningitidis
MKPLQMLPIAALVGSIFGNPVLAADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYNHHALGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWRVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKVKDAQYTVYENKGWGTPLQKKVKDYPWLNKSAYVFDMYGFYKPAKNLTLRAGVYNLFNRKYTTWDSLRGLYSYSTTNAVDRDGKGLDRYRAPGRNYAVSLEWKF

SEQ ID NO: 14 - protein, 591 aa, Neisseria meningitidis
MNKIYRIIWNSALNAWVVVSELTRNHTKRASATVKTAVLATLLFATVQASANNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLPGKSMMAIGGGTYRGEAGYAIGYSSISDGGNWIIKGTASGNSRGHFGASASVGYQW

SEQ ID NO: 15 - protein, 1457 aa, Neisseria meningitidis
MKTTDKRTTETHRKAPKTGRIRFSPAYLAICLSFGILPQAWAGHTYFGINYQYYRDFAENKGKFAVGAKDIEVYNKKGELVGKSMTKAPMIDFSVVSRNGVAALVGDQYIVSVAHNGGYNNVDFGAEGRNPDQHRFTYKIVKRNNYKAGTKGHPYGGDYHMPRLHKFVTDAEPVEMTSYMDGRKYIDQNNYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPRQNGKYSFNDDNNGTGKINAKHEHNSLPNRLKTRTVQLFNVSLSETAREPVYHAAGGVNSYRPRLNNGENISFIDEGKGELILTSNINQGAGGLYFQGDFTVSPENNETWQGAGVHISEDSTVTWKVNGVANDRLSKIGKGTLHVQAKGENQGSISVGDGTVILDQQADDKGKKQAFSEIGLVSGRGTVQLNADNQFNPDKLYFGFRGGRLDLNGHSLSFHRIQNTDEGAMIVNHNQDKESTVTITGNKDIATTGNNNSLDSKKEIAYNGWFGEKDTTKTNGRLNLVYQPAAEDRTLLLSGGTNLNGNITQTNGKLFFSGRPTPHAYNHLNDHWSQKEGIPRGEIVWDNDWINRTFKAENFQIKGGQAVVSRNVAKVKGDWHLSNHAQAVFGVAPHQSHTICTRSDWTGLTNCVEKTITDDKVIASLTKTDISGNVDLADHAHLNLTGLATLNGNLSANGDTRYTVSHNATQNGNLSLVGNAQATFNQATLNGNTSASGNASFNLSDHAVQNGSLTLSGNAKANVSHSALNGNVSLADKAVFHFESSRFTGQISGGKDTALHLKDSEWTLPSGTELGNLNLDNATITLNSAYRHDAAGAQTGSATDAPRRRSRRSRRSLLSVTPPTSVESRFNTLTVNGKLNGQGTFRFMSELFGYRSDKLKLAESSEGTYTLAVNNTGNEPASLEQLTVVEGKDNKPLSENLNFTLQNEHVDAGAWRYQLIRKDGEFRLHNPVKEQELSDKLGKAEAKKQAEKDNAQSLDALIAAGRDAVEKTESVAEPARQAGGENVGIMQAEEEKKRVQADKDTALAKQREAETRPATTAFPRARRARRDLPQLQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTENTFDDGIGNSARLAHGAVFGQYGIDRFYIGISAGAGFSSGSLSDGIGGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 16 - protein, 797 aa, Neisseria meningitidis
MKLKQIASALMMLGISPLALADFTIQDIRVEGLQRTEPSTVFNYLPVKVGDTYNDTHGSAIIKSLYATGFFDDVRVETADGQLLLTVIERPTIGSLNITGAKMLQNDAIKKNLESFGLAQSQYFNQATLNQAVAGLKEEYLGRGKLNIQITPKVTKLARNRVDIDITIDEGKSAKITDIEFEGNQVYSDRKLMRQMSLTEGGIWTWLTRSNQFNEQKFAQDMEKVTDFYQNNGYFDFRILDTDIQTNEDKTKQTIKITVHEGGRFRWGKVSIEGDTNEVPKAELEKLLTMKPGKWYERQQMTAVLGEIQNRMGSAGYAYSEISVQPLPNAETKTVDFVLHIEPGRKIYVNEIHITGNNKTRDEVVRRELRQMESAPYDTSKLQRSKERVELLGYFDNVQFDAVPLAGTPDKVDLNMSLTERSTGSLDLSAGWVQDTGLVMSAGVSQDNLFGTGKSAALRASRSKTTLNGSLSFTDPYFTADGVSLGYDVYGKAFDPRKASTSIKQYKTTTAGAGIRMSVPVTEYDRVNFGLVAEHLTVNTYNKAPKHYADFIKKYGKTDGTDGSFKGWLYKGTVGWGRNKTDSALWPTRGYLTGVNAEIALPGSKLQYYSATHNQTWFFPLSKTFTLMLGGEVGIAGGYGRTKEIPFFENFYGGGLGSVRGYESGTLGPKVYDEYGEKISYGGNKKANVSAELLFPMPGAKDARTVRLSLFADAGSVWDGKTYDDNSSSATGGRVQNIYGAGNTHKSTFTNELRYSAGGAVTWLSPLGPMKFSYAYPLKKKPEDEIQRFQFQLGTTF

SEQ ID NO: 17 - protein, 180 aa, Neisseria meningitidis
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQR

SEQ ID NO: 18 - protein, 644 aa, Neisseria meningitidis
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 19 - protein, 350 aa, Neisseria meningitidis
MKHFPSKVLTTAILATFCSGALAATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 20 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDLGGEHTSFDKLPEGGRATYRGTAFGSDDAGGKLTYTIDFAAKQGNGKIEHLKSPELNVDLAAAEIKADEKSHAVILGDVRYNQAEKGTYSLGIFGGKAQEVAGSAEVKTVNGIRHIGLAAKQ

SEQ ID NO: 21 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYHGKAFGSDDPNGRLHYTIDFAAKQGYGRIEHLKTPEQNVDLAAADIKPDGKRHAVISGSVLYNQAEKGSYSLGIFGGKAQEVAGSAEVKIGEGIRHIGLAAKQ

SEQ ID NO: 22 - protein, 247 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDLGGEHTAFNQLPDGKAEYRGTAFGSDDAGGKLTYTIDFTKKQGNGKIEHLKSPELNVELASAEIKADGKSHAVILGDVRYGSEEKGSYSLGIFGGRAQEVAGSAEVKTVNGIRHIGLAAKQ

SEQ ID NO: 23 - protein, 248 aa, Neisseria meningitidis
VAADIGAGLADALTAPLDHKDKGLQSLTLDQSVRKAEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 24 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTAQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 25 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMAAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 26 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDALPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 27 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVAYNQAEKGSYSLGIFGGKAQEVAGSAEVKTVNGIRHIGLAAKQ

SEQ ID NO: 28 - protein, 248 aa, Neisseria meningitidis
VAADIGAGLADALTAPLDHKDKGLQSLTLGQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDLGGEHTSFDKLPEGGRATYRGTAFGSDDAGGKLTYTIDFAAKQGNGKIEHLKSPELNVDLAAAEIKADEKSHAVILGDVRYNQAEKGTYSLGIFGGKAQEVAGSAEVKTVNGIRHIGLAAKQ

SEQ ID NO: 29 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGAATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 30 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATARGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 31 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYAGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 32 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAAQ

SEQ ID NO: 33 - DNA, 26 bp, Artificial Sequence
  Immunostimulatory oligonucleotide
ncncncncncncncncncncncncnc

SEQ ID NO: 34 - protein, 11 aa, Artificial Sequence
  Polycationinc oligopeptide
KLKLLLLLKLK

SEQ ID NO: 35 - protein, 46 aa, Artificial Sequence
  N-terminus sequence from Figure 6 of WO2010/046715
MPSEPPFGRHLIFASLTCLIDAVCKKRYHNQNVYILSILRMTRSKP